(19) United States
(12) Patent Application Publication
(10) Pub. No.: US 2018/0318610 A1
(43) Pub. Date: Nov. 8, 2018

(54) Disruption of tau entanglements and amyloid plaques in the human brain by low frequency high power focused ultrasound

(71) Applicant: Jaan Noolandi, La Jolla, CA (US)

(72) Inventor: Jaan Noolandi, La Jolla, CA (US)

(21) Appl. No.: 15/965,868

(22) Filed: Apr. 28, 2018

Related U.S. Application Data
(60) Provisional application No. 62/501,177, filed on May 4, 2017.

(51) Int. Cl. A61N 7/00

(52) U.S. Cl. CPC A61N 7/00; A61N 2007/0021

(57) ABSTRACT

A method is provided to disrupt tau entanglements and amyloid plaques in a person's brain. An ultrasound profile generated by an ultrasound device is applied to the person's brain. The generated ultrasound profile is characterized by a frequency between 20-100 kHz, a pulse power greater than 10 W/cm⁻², a pulse length greater than 1 microsecond, and an interval between pulses greater than 1 millisecond. The method is performed without intravenous injection of microbubbles to the person or use of preformed microbubbles.

### CROSS-REFERENCE TO RELATED APPLICATIONS

This application claims priority from U.S. Provisional Patent Application 62/501,177 filed May 4, 2017, which is incorporated herein by reference.

### FIELD OF THE INVENTION

The invention relates to methods for disrupting plaques and neurofibrillary tangles in the human brain.

### BACKGROUND OF THE INVENTION

Modern techniques like magnetic resonance imaging (MRI) have identified certain abnormal structural features, such as amyloid-β (Aβ) plaques, in the brains of patients suffering from diseases such as Alzheimer's. One noninvasive method of studying and treating these features is the use of focused ultrasound. Focused ultrasound in the MHz frequency range following the intravenous injection of microbubbles has been used for drug delivery across the blood-brain barrier to target, in particular, the amyloid-β (Aβ) plaques. As the microbubbles pass through blood vessels at the focus of the ultrasound they expand and contract in response to the propagating ultrasound wave, stimulating the opening of the blood-brain barrier and allowing therapeutic agents to enter the brain parenchyma. Low frequency non-focused ultrasound (20-30 kHz) coupled to a scanning device with a pinhole has also been used to disrupt the blood-brain barrier using preformed microbubbles.

Other neurotoxic elements in the human brain are neurofibrillary entangles and fibrous aggregates of tau, a tubulin-binding protein that stabilizes microtubules in neurons. Various novel drugs have been injected across the blood-brain barrier using focused ultrasound to target neurofibrillary entangles and inhibit tau aggregation.

The effectiveness of ultrasound using preformed microbubbles to open the blood-brain barrier is open for debate. For example, one approach is magnetic resonance-guided focused ultrasound with a phased array of transducers. The phased array of transducers allows for ultrasound pulses to be delivered to the same spot at timed intervals to avoid overheating. Along with computed tomography data of the human skull, this method has the advantage of targeting acoustic energy into small volumes at specific sites. The usefulness of the technique relies on the development of real-time acoustic feedback control systems to determine safe but adequate ultrasound exposures. Currently, such systems have been employed only in the high frequency regime.

To date there has been limited success to dissolve or limit the growth of extracellular Aβ plaques and intracellular tau neurofibrillary tangles using focused ultrasound opening of the blood-brain barrier and the therapeutic value of the technique remains to be determined. The present invention addresses this limited success by providing a new approach to dissolve and/or limit the growth of extracellular Aβ plaques and intracellular tau neurofibrillary tangles.

### SUMMARY OF THE INVENTION

The present invention provides a method and system using low frequency (20 to 100 kHz) and high-power intensity (greater than 10 Wcm⁻²) focused ultrasound, without the use of preformed microbubbles, to disrupt tau protein entanglements, neurotoxic insoluble aggregates and amyloid-β plaques in the human brain.

Macromolecules can be cleaved by using low frequency ultrasound regime (20 to 100 kHz) above a threshold value of power and above a critical molecular weight for the macromolecules. The polymer degradation process results from the creation of transient cavitation microbubbles, which are vapor-filled voids, produced using power intensities in excess of 10 Wcm⁻². Their violent collapse, on the order of microseconds, generates shear forces, which can cause the entangled polymer chains and aggregates to stretch and destruct when the tension overcomes the strength of molecular bonds.

The critical molecular weight of the polymers is related to the entanglement molecular weight, when a fixed number of polymer chains cohabit the same volume. The theory of polymer entanglements states that at a given polymer concentration the motion perpendicular to the polymer backbone is quenched. Once this concentration is reached the entanglements are permanent in the sense that the polymers can only move by thermal motion in tortuous paths along their backbones by a process called reptation. This is a very slow process and no physical mechanism by itself can untangle the molecules in a polymer melt. In effect the entangled polymers and aggregates are fixed entities. Short chains, which are not immobilized by their entangled or cross-linked environment, may not be subjected to a sufficient shear rate during the ultrasound pulse to cause breakage and structures which are softer and more mobile may respond to the collapsing cavitation microbubbles by deformation rather than destruction.

Outside of the low frequency as used in the embodiments of this invention, high power regime of ultrasound the cavitation process becomes less violent and in the high frequency regime (100 kHz-2 MHz) little or no polymer degradation is observed. Hence the low frequency and high energy regime of ultrasound as provided in this invention is preferred to disrupt neurotoxic structural features in the human brain.

At the same time the relatively long wavelengths of low frequency ultrasonic waves result in lower spatial resolution, presenting a challenge for therapeutic applications. The use of metamaterials addresses the issue of increasing the resolution of focused low frequency ultrasound.

Since the bubble cavitation process takes place in microseconds, a low frequency ultrasound pulse of the order of a second (or less) focused regularly on the same spot every few seconds is enough to disrupt entanglements and other rigid structures. Although for synthetic polymers a power density of 10 W cm⁻² for ultrasound pulses of 20 kHz is sufficient to cause chain cleavage, higher power densities may be necessary for the proposed application to compensate for the absorption and scattering of the sound waves as they pass through the human skull.

Pulsed low frequency high power ultrasound as defined herein could be clinically effective in disrupting rigid structures like cross-linked tau protein aggregates, tight entanglements and amyloid-β plaques. For reasons of safety the detailed protocols and computer algorithms for the power, the frequency of the pulse as well as the duration of the pulse and their repeat frequency must be monitored to avoid tissue damage and intracranial overheating.

In one embodiment, the invention can be characterized as a method to disrupt tau entanglements and amyloid plaques in a person's brain. In this method an ultrasound profile generated by an ultrasound device is applied to the person's brain. The generated ultrasound profile applied to the person's brain is characterized by:
(i) a frequency between 20-100 kHz,
(ii) a pulse power greater than 10 W/cm⁻²,
(iii) a pulse length greater than 1 microsecond, and
(iv) an interval between pulses greater than 1 millisecond.

In another embodiment, the invention can be characterized as a method (wherein the improvement comprises) to disrupt tau entanglements and amyloid plagues in a person's brain with the application of the same ultrasound profile to the person's brain as taught herein in this invention.

In still another embodiment, the invention can be characterized as a method to disrupt tumors in a person's brain with the application of the same ultrasound profile to the person's brain as taught herein in this invention.

In all embodiments of this invention, the method is performed without intravenous injection of microbubbles to the person or use of preformed microbubbles. Furthermore, in some embodiments the ultrasound profile is focused using metamaterials.

### BRIEF DESCRIPTION OF THE DRAWINGS

FIGS. 1A-B show according to an exemplary embodiment of the invention the concept of super-resolution ultrasound focusing by using an artificial metamaterials based super-lens and showing how to use this method for the treatment of neurotoxic features in the human brain. FIG. 1A shows schematics of a metamaterial based super-lens helmet system to focus ultrasound into the human brain. The 3D location of the focal spot can be controlled by scanning the acoustic point source outside of the helmet. FIG. 1B shows rectangular slab of a negative index metamaterial can act as a near-perfect lens, leading to a super resolution image of the point source on the other side of the slab.

### DETAILED DESCRIPTION

Low frequency ultrasonic waves (20 kHz-100 kHz) have relatively long wavelength (75 nm-15 mm in soft tissue), which makes it difficult to focus into a small spot. In an embodiment of this invention an artificial material is used to make a lens, i.e. metamaterial super-lens, to achieve super-resolution focusing of ultrasound pulses in the human brain. Metamaterials are artificial materials composed of deep-subwavelength scale structures, leading to extraordinary material properties that do not exist in nature. Metamaterials has been a popular research field in physics and engineering within the last 1-2 decades. Novel applications, such as deep-subwavelength imaging has been achieved by using metamaterial lenses. In this invention, we introduce a paradigm shifting technology to project focused ultrasonic sound from outside into the human brain with the help of a metamaterial super-lens helmet. The projected image (focused ulatrasound spot) possesses super resolution (mm or sub-mm scales) and its position can be controlled by tuning the location of the ultrasound source. High power tightly focused low frequency ultrasound, combined with an appropriate scanning pattern, can then be used to treat the features that are thought to be responsible for Alzheimer's disease or features associated with other (brain) diseases.

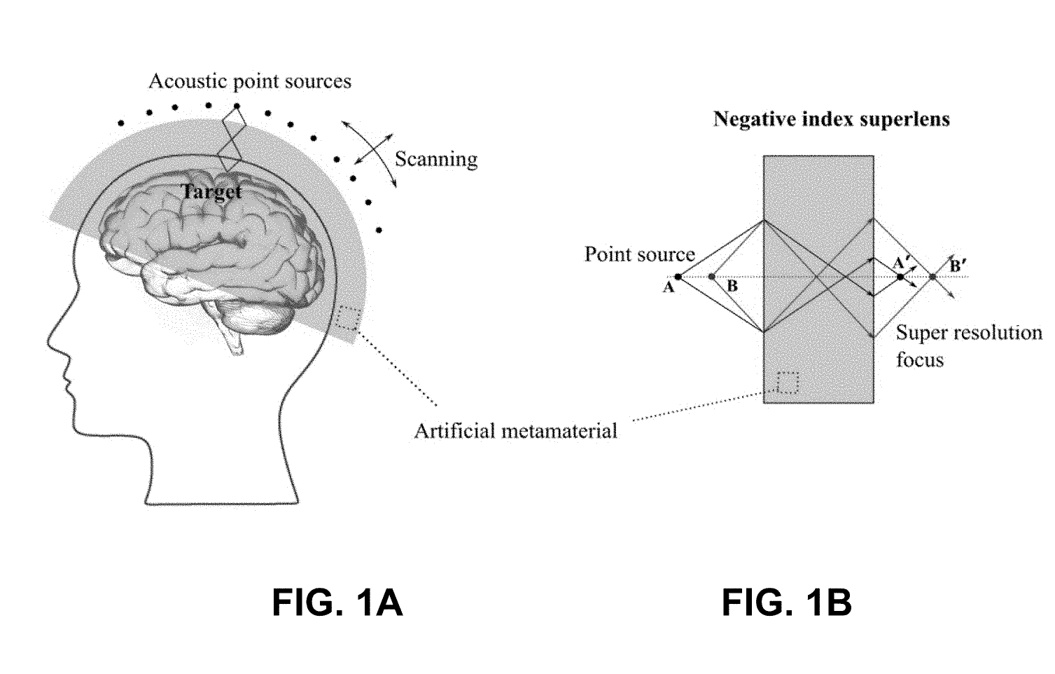

To focus ultrasound inside the skull in the brain area with super resolution, as shown in FIGS. 1A-B, a super-lens helmet is used made out of a specially designed metamaterial to go beyond the diffraction limit. Normally one cannot focus ultrasound to a spot with size less than half the wavelength of ultrasound due to the fading evanescent fields which carry the subwavelength features of objects. By focusing the propagating wave and also recovering the evanescent field, a super-lens with a negative index can overcome the diffraction limit and bring the wave into a super-resolution focus.

As shown in FIG. 1B, when an ultrasound wave from a point source strikes a negative-index metamaterial the flat interface bends the wave to a negative angle with the surface normal to focus once inside the lens and once outside the lens resulting in two focused images. More importantly, the typically fading evanescent field would be significantly amplified by the negative-index material, allowing for subwavelength resolution focus. Similar to traditional lenses, the change of object-lens distance will lead to a change of the image-lens distance (FIG. 1B), which enables arbitrary positioning of the focal spot in 3D space by simply scanning the position of the point source.

Modern metamaterials are made from assemblies of multiple elements fashioned from composite materials such as metals or plastics and are arranged in repeating patterns, at scales that are much smaller than the wavelengths of the incident ultrasound, with a negative index. An acoustic metamaterial can possess simultaneously negative bulk modulus and mass density by combining several types of structural elements, enabling the super-lensing functions.

Metamaterials are artificial structures, typically periodic (but not necessarily so), composed of small building units that, in the bulk, behave like a continuous material with extraordinarily effective properties. By designing and engineering each unit cell of a periodic structure, often referred to as a meta-atom, negative values of effective mass density and bulk modulus can be obtained, thus offering negative refraction. Such materials allow for the guiding and focusing of acoustic waves far beyond the diffraction limit. For our focused ultrasound applications, the negative index super-lens can be achieved through several approaches. First, a single resonator can have multiple eigen-modes exhibiting distinctive symmetries. By careful design, it is possible to tune the frequencies of these eigen-modes to our range of interest and realize simultaneously negative density and bulk modulus values. Alternatively, combining two different resonating structures, each having one type of symmetry, can also lead to double negativity. Space-coiling structures also give rise to double-negative metamaterials. Different from the former two, space-coiling structures are based on the design of the “acoustic path” to modulate the phase to achieve negative refraction. The space-coiling based metamaterials can be directly prepared by using plastic, which is easy to design and fabricate.

What is claimed is:

1. A method to disrupt tau entanglements and amyloid plaques in a person's brain, comprising:
applying an ultrasound profile to the person's brain generated by an ultrasound device, wherein the generated ultrasound profile applied to the person's brain is characterized by:
(i) a frequency between 20-100 kHz,
(ii) a pulse power greater than 10 W/cm⁻²,
(iii) a pulse length greater than 1 microsecond, and
(iv) an interval between pulses greater than 1 millisecond,
wherein the method is performed without intravenous injection of microbubbles to the person or use of preformed microbubbles.

2. The method as set forth in claim 1, wherein the ultrasound profile is focused using metamaterials.
